(19) United States
(12) Patent Application Publication
(10) Pub. No.: US 2007/0275096 A1
(43) Pub. Date: Nov. 29, 2007

(54) Composition and methodology for the treatment of hearing impairment symptoms

(75) Inventor: Brian Banks, Dillon, CO (US)

Correspondence Address: John D. Long, PATENT COUNSEL;LONG & CHYBIK, Suite 115, 1575 Delucchi Lane, Reno, NV, 89502, US

(73) Assignee: TRP, Inc., a Nevada Corporation, Reno, NV (US)

(21) Appl. No.: 11/805,116

(22) Filed: May 21, 2007

Related U.S. Application Data
(60) Provisional application No. 60/802,241, filed on May 19, 2006.

(51) Int. Cl. A61K 36/38; A61K 36/28; A61K 31/21; A61K 33/06; A61K 33/28

(52) U.S. Cl. 424/644; 424/730; 424/764; 424/724; 424/696; 514/159; 514/514

(57) ABSTRACT

One possible embodiment of the invention could include a homeopathic composition suitable for the treatment of the symptoms of hearing impairment comprises: 3-Isothiocyanato-1-Propene in 6 times potency, Arnica Montana in 6 times potency, Arnica Montana in 30 times potency, Calcium Sulphide in 6 times potency, Hypericum Perforatum in 6 times potency, Hypericum Perforatum in 30 times potency, Silicon Dioxide in 13 times potency, Mercury (Hg) in 13 times potency, and Salicylic Acid in 6 times potency. The embodiment may further comprise delivery of the above composition in sufficient homeopathic quantity to the afflicted ear(s).

### CROSS-REFERENCES TO RELATED APPLICATIONS

This present application claims the benefit under 35 U.S.C. § 119(e) of U.S. Provisional Patent Application No. 60/802241, filed on May 19, 2006, contents of which are relied upon and incorporated by reference.

### STATEMENT REGARDING FEDERALLY SPONSORED RESEARCH OR DEVELOPMENT

Not Applicable

### REFERENCE TO A “MICROFICHE APPENDIX”

Not Applicable.

1. Field of the Invention

The present invention generally relates to a composition for the treatment of symptoms for hearing impairments. In particular, to the hearing impairments that may be due to aging.

2. Background

As a person ages, they may notice the arising of one or more impairments to their hearing. Although such impairment could occur in a single instance, [e.g. experiencing, without proper sound protection, an air compression wave or sound from a powerful source (e.g., industrial accident or combat experience)], many aging baby boomers over a significant portion of their life time may have chronically listened to music and other sounds for long periods at time at a high volume, often employing a headset or ear phones. This pattern of conduct may result in or possibly acerbate a predisposition (e.g., due to genetics, aging and the like) for sensorineural hearing loss(es). Sensorineural hearing loss(es) may include hearing impairment regarding the sound's intensity or loudness (measured in units called decibels, dB) (e.g., a person could no longer hear soft sounds, only loud ones); the sound's frequency or pitch (measured in units called hertz, Hz) (e.g., hearing low notes but not high notes); or both.

Such conduct may result in sensorineural hearing impairment that could be related to changes in the inner ear. The inner ear may comprise the cochlea, a generally hollow coil shaped organ, which may translate the physical disturbances in the air (as captured by the ear drum and then transmitted by ossicles [three tiny bones, the malleus, incus and stapes] into the interior of the cochlea) into electro-neural pulses that are then transmitted to and substantially processed by the brain to generally create the sensation of sound. The cochlea may be comprised of two portions, a coil portion and a multiple loop portion. The multiple loop portion may comprise of semicircular canals, which are used to give us a sense of balance and orientation.

The coil portion, which is a part of the body's hearing apparatus, generally has a hollow, fluid-filled interior that may be further lined with ciliated cells (cells having hair-like protrusion) appropriately enough known as hair cells. The hair-like protrusion generally leaves the hair cell to connect substantially to a membrane within the fluid-filled interior of the cochlea. When the physical movement is substantially received from the ear drum and mechanically transmitted to the interior of the cochlea, the cochlea's interior fluid may be displaced to move generally the membrane. The moving membrane then in turns substantially moves the cilia of the respective hair cell causing the respective hair cell to create substantially an electrical impulse that is eventually transmitted by the auditory nerve to the brain for processing as sound sensation.

One theory of sound, the place theory, generally sets forth that zones or placement of hair cells corresponding with hearing sensation of certain sound frequencies (e.g., the tip of the cochlea coil handles high frequencies-high notes while zones progressing to the base of the cochlea progressively handling lower frequencies-low notes). So that during aging process, for example, a hearer's higher frequency zones may suffer more damage to the cilia cells than those located in the lower frequency zones causing the hearer to miss out on high notes (e.g., a women's voice with a higher sound register range) but able to better hear lower notes (e.g., a man's voice with deeper note range). Once the cilia of the hair cells are damaged they generally do not grow back.

Other hearing loss types may include a conductive hearing loss that may be caused by diseases or obstructions in the outer or middle ear (e.g., the conduction pathways for air waves to reach the inner ear) and a central hearing loss that may result from damage or impairment to the nerves or nuclei of the central nervous system, either in the pathways to the brain or in the brain itself.

Sometimes the sensation of a constant or a semi-continous noise or ringing in the ear is also associated with such hearing impairment. This ringing or tinnitus may be a long term or temporary affliction. Temporary causes may include waxy build up in the ear canal, infection, certain conventional medications, temporary exposure to excessive noise, and the like. Long term occurrence may be the result of damaged cilia of the hair cells (e.g. aging, being subjected to continuous loud noise for long period of time, illness, etc); jaw misalignment, cardiovascular disease, certain types of tumors, head and neck trauma, and the like.

Conventional medicine may attempt to rectify such hearing loss through sound amplification systems such as hearing aids, cochlear implants as well as through conventional pharmaceutical intervention.

In addition to the above conventional medical remedies, there is a continued consumer interest in the use and development of complementary medicines that are otherwise generally underutilized by the conventional medicine practitioners. Typically, complementary medicine may be used to supplement the primary or conventional medical treatments. One such field of complementary medicine is the homeopathy, which may use medicine formulations or remedies that have ingredients that are generally not used in conventional medicine. Further, such homeopathic remedies may present their active ingredients in concentrations much lower than the concentrations substantially used in conventional medicine formulations.

Homeopathy was first developed 1790 where it was generally noticed that a prevalent treatment for malaria at that time was ingestion of bark of the cinchona tree (much later, quinine, a major conventional medicine anti-malarial drug, was substantially extracted and refined into higher concentrations from this bark). At that time, conventional medicine generally held that the bark's effectiveness was due to its very bitter taste. It was then generally noticed other medicines were equally bitter, but they were not particularly useful in the treatment of malaria, and that other aspects of this remedy must be responsible for its medicinal properties. It was substantially discovered that a person not having the corresponding disease (e.g., malaria) took (e.g., ingested) of the remedy (e.g., the cinchona bark) then that person began to exhibit all the disease's (e.g., malaria) symptoms (e.g., chills, fever, palpitations, sweats, etc). It was further rationalized that it could be possible to develop generally a remedy for an illness based on those elements or ingredients, which mimicked the symptoms of the disease when taken by a healthy person. The remedy, however, could contain dosages of the selected element(s) in quantity(ies) much lower than those levels that caused the symptoms in otherwise healthy individuals taking the selected elements.

Practitioners of Homeopathy (e.g., Homeopaths) used the above homeopathic remedy development doctrine to individually identify various potential homeopathic elements. These provings (e.g. testing and recording of the results for various potential homeopathic ingredients or elements) were then collected and organized in various texts. One such text, the Homeopathic Pharmacopoeia of the United States (HPUS), is used by the US Food and Drug Administration (FDA) as its official homeopathic remedy guideline for the classification and regulation of the manufacture homeopathic remedies or medicines. Today's Homeopath will use the HPUS to select substantially one or more approved ingredients at regulated levels to form a homeopathic remedy.

Many times, the source of the ingredients for a homeopathic remedy could be a naturally occurring organic substance such as an herb or the like. In such cases, the succus (e.g., an extract) could be produced from herb or the like by taking a sample of the substance and squeezing or otherwise removing the juice or the extract of the ingredient source. In some cases, the succus would be further titrated [e.g., preparing the smallest amount of an element of known concentration that is required to bring about a given effect (e.g., replicate illness symptoms) in reaction in a patient)] to form a tincture.

The tincture then could be further diluted by factors of 10 with an HPUS approved diluent. For example, a 10² dilution factor could be made by substantially having one part of the succus or its tincture placed into 100 parts of a diluent (e.g., purified water) (e.g., 1/10²). The resulting diluted volume could then go through a “potentizing” wherein then diluted volume could be place and sealed into a heavy glass jar for further mixing. The homeopathic remedy preparer could then lift the sealed heavy glass jar up above his or her head and then slap the jar down onto a padded surface (e.g., folded-over leather pad) several times. This potentizing may then induce an agitation or turbulence in the diluted volume, which is believed to mix properly a tincture/a otherwise diluted solution into the dilution volume (e.g., the current diluent).

The dilution process could be repeated again (for another 1× dilution) and be generally followed by a respective potentizing action to achieve substantially the HPUS prescribed ultimate dilution for the selected element. Once each of the various active ingredients or elements are made to their respective proper dilutions, they could be placed in an appropriate delivery matrix (e.g., a saline solution containing appropriate preservative, gel base for a salve, etc) to form the homeopathic remedy to be given to the patient. The selection of the deliver matrix and amounts of properly diluted homeopathic elements placed in the matrix could be accomplished by a person having ordinary knowledgeable in the art.

In using the homeopathic remedy analysis, the homeopath generally has to decide on what observable indicators present in the sick patient correlates the respective symptoms of an imbalance of the body or disease. The homeopath, as a diagnostician, generally examines a wider field of symptoms (e.g. the emotional state of the patient and the like) than does conventional medical practitioner. Once the present indicators of the patent are properly identified as illness symptoms, the homeopath then tries to match those identified symptoms to those symptoms caused by homeopathic ingredients as documented in the HPUS. In selecting the proper active ingredients or elements for the homeopathic remedy, homeopath may take into account that some homeopathic ingredients may mimic one or more illness symptoms.

What is needed therefore is a homeopathic remedy that can be used to aid the relief of one or more symptoms accompanying the infirmities of the ear, such as those which may accompany a person as they age.

### SUMMARY OF ONE EMBODIMENT OF THE INVENTION

Advantages of One or More Embodiments of the Present Invention

The various embodiments of the present invention may, but do not necessarily, achieve one or more of the following advantages:

the ability to possibly alleviate or otherwise ameliorate one or more symptoms associated with frequency-based sensorineural hearing loss;

the ability to possibly alleviate or otherwise ameliorate one or more symptoms associated with intensity-based sensorineural hearing loss;

the ability to possibly alleviate or otherwise ameliorate one or more symptoms associated with damage or other wise impaired cilia of the hair cells; and

the ability to possibly alleviate or otherwise ameliorate one or more symptom associated with tinnitus.

These and other advantages may be realized by reference to the remaining portions of the specification, claims, and abstract.

### BRIEF DESCRIPTION OF THE DRAWINGS

FIG. 1 is substantially a list of elements or ingredients comprising the homeopathic composition for the invention.

### BRIEF DESCRIPTION OF ONE EMBODIMENT OF THE PRESENT INVENTION

In one aspect, the invention could be an composition to possibly alleviate or otherwise mitigate one or more symptoms of hearing impairment associated with aging. substantially comprising of at least the diluted tinctures of several homeopathic ingredients, namely (1) Thiosinaminum (i.e., the herb Oil of mustard seed), (2) Arnica Montana (i.e., the herb Leopards Bane), (3) Hepar sulph calc (Calcium Sulphide), (4) Hypericum Perforatum (i.e., the herb St. John's Wort) (5) Silicon dioxide (SiO₂), (6) Mercury (Hg), and (7) Salicylic Acid [C6H4(OH)COOH].

In another aspect, the invention could be a method of treating hearing distress or impairment in a human comprising administrating to a human a homeopathic effective amount of the aforesaid homeopathic composition.

The above description sets forth, rather broadly, a summary of one embodiment of the present invention so that the detailed description that follows may be better understood and contributions of the present invention to the art may be better appreciated. Some of the embodiments of the present invention may not include all of the features or characteristics listed in the above summary. There are, of course, additional features of the invention that will be described below and will form the subject matter of claims. In this respect, before explaining at least one preferred embodiment of the invention in detail, it is to be understood that the invention is not limited in its application to the details of the construction and to the arrangement of the components set forth in the following description. The invention is capable of other embodiments and of being practiced and carried out in various ways. Also, it is to be understood that the phraseology and terminology employed herein are for the purpose of description and should not be regarded as limiting.

### DESCRIPTION OF CERTAIN EMBODIMENTS OF THE PRESENT INVENTION

In the following detailed description of the preferred embodiments, specific embodiments may be set fourth in which the invention may be practiced. It is to be understood that other embodiments may be utilized and structural changes may be made without departing from the scope of the present invention.

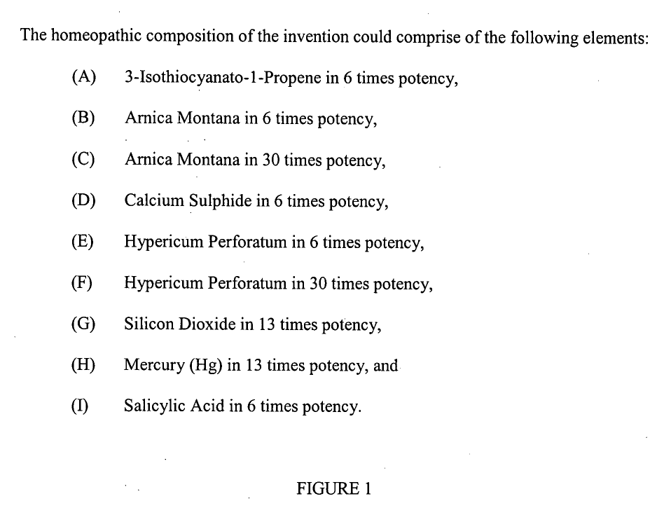

As substantially shown in FIG. 1, the present invention could be a homeopathic composition comprising of the following active ingredients namely (1) Thiosinaminum (oil of mustard seed), (2) Arnica Montana (Leopards bane), (3) Hepar sulph calc (calcium sulphide), (4) Hypericum (St. John's-wort) (5) Silicea (silicon dioxide), (6) Mercury, and (7) Salicylic Acid [C6H4(OH)COOH] which are available from Apothecara Natural, Inc. of Woodbine, Iowa, USA.

In one possibly embodiment, the elements or active ingredients of the above-composition could be diluted by standard homeopathic methods wherein a 1× dilution would mean taking 1 part by weight of an active homeopathic ingredient and diluting into 10 parts by weight of diluent (e.g., filtered water or the like) to create a 10% by weight dilution of the initial homeopathic amount. Similarly, a 2× dilution would be taking 1 part by weight of 1× dilution and placing it into 10 parts of diluent to create a 3× or 1% by weight dilution. A 1 part by weight of the 3× dilution could then placed in a 10 parts by weight diluent to create 4× or 0.1% by weight dilution. In this manner, the NX homeopathic dilution indicates that the dilution procedure had occurred N times for that active homeopathic ingredient or element (e.g., N generally being a whole number.) Each dilution would be followed by a respective potentizing. The accomplished dilutions could vary with a tolerance of 10%.

In at least one embodiment, the homeopathic composition could be administered to the ear canal of the subject via a liquid format (e.g., drop or stream). Said liquid format (e.g., matrix, carrier, etc.) in addition to containing or incorporating the appropriate amounts of the homeopathic active ingredients, could also contain inactive ingredients glycerin [sometimes used for softening (for removal) of excess wax build up] and polysorbate solution with preservatives and the like as known by those skilled in the art for use in ear drop medication, remedies, and the like. Another possible carrier or a matrix that could be used in conjunction with the homeopathic composition could be propylene glycol vehicle with 3% propylene glycol diacetate with benzalkonium chloride. As such, one possible embodiment of the invention, the homeopathic composition of the above-named seven (7) active homeopathic ingredients could be generally contained in a liquid format of glycerin solution or propylene glycol matrix or carrier for delivery to the afflicted ear.

The concentrations of the active homeopathic ingredients of the homeopathic composition of the invention could be as follows:

The element Thiosinaminum or 3-Isothiocyanato-1-Propene (i.e., NH₂CSNHCH₂CH₂) (also known as oil of mustard seed) could be initially prepared by warming a mixture of equal parts of allyl mustard oil and absolute alcohol with an equal amount of 30 per cent ammonia. A HPUS approved tincture could be made by taking 100 grams of initial preparation and combining it with 900 grams lactose or milk sugar to form generally one thousand grams of Thiosinaminum titration. The HPUS approved tincture could then be further diluted with a diluent of 90% glycerin/10% purified water.

The tincture could be diluted six times utilizing the 1× dilution technique (with accompanying potentization) for inclusion into the present homeopathic composition. The resulting dilution could be a 6× factor dilution (e.g., 1/10⁶ dilution; a 1 part tincture per 1,000,000 parts diluent of 90% glycerin/10% purified water solution.) With proper occupying potency, the amount of the Thiosinaminum (e.g., 3-Isothiocyanato-1-Propene , NH₂CSNHCH₂CH₂, Oil of Mustard Seed) in the homeopathic composition of the present invention could be at six times potency.

The herb Arnica Montana (Leopards bane) could be prepared into a HPUS approved tincture, made from the whole fresh plant. This tincture could be accomplished using 100 grams of powderized Arnica Montana combined with 400 ml of purified water and 635 ml of strong alcohol to make substantially the tincture. This HPUS tincture could then be further diluted with a diluent of 90% glycerin/10% purified water.

For inclusion into the homeopathic composition of the present invention, the tincture could be further diluted six times utilizing the 1× dilution technique (with accompanying potentization) substantially using a HPUS approved diluent of 90% glycerin/10% purified water solution. The resulting dilution could be a 6× dilution (e.g., a 1/10⁶ dilution or a 1 part tincture per 1,000,000 parts of diluent.) With proper accompanying potency, Arnica Montana could be present in the homeopathic composition at six times potency.

The herb Arnica Montana could also be again present in the homeopathic composition at 30 times potency using the above described methodology of dilution.

The homeopathic element Calcium Sulfide (CaS) could be titrated into a HPUS approved tincture utilizing 100 grams of coarse powder of calcium sulfide in a amorphous powder form and mixing it into 900 grams of lactose (milk sugar) to make substantially a thousand grams of the Mother Tincture. This tincture could then be diluted with a HPUS-approved diluent of 90% glycerin/10% purified water.

The tincture in the present invention could be diluted six times utilizing a 1× dilution technique (with accompanying potentization) using the HPUS-approved diluent of 90% glycerin/10% purified water. The resulting dilution as used in the homeopathic composition of the invention could be a 6× dilution factor ( 1/10⁶ dilution or 1 part tincture per 1,000,000 parts diluent). Calcium sulfide could be at six times potency for the homeopathic composition.

The herb Hypericum Perforatum (i.e., St. John's-Wort) could be prepared into a HPUS-approved tincture utilizing the whole fresh plant/root. The Mother Tincture of the herb could then be diluted six times utilizing HPUS approved diluent of 90% glycerin/10% purified water solution with a 1× dilution technique (with accompanying potentization). The result could be a 6× dilution factor ( 1/10⁶ dilution) with a resulting dilution substantially being a 1 part tincture per 1,000,000 parts per approved diluent. The amount of Hypericum Perforatum in the homeopathic composition of the invention could be six times potency.

The herb Hypericum Perforatum (i.e., St. John's-Wort) could again be present in the homeopathic composition in thirty (30) times potency. This being thirty times potency could be obtained with a dilution factor ( 1/10³⁰ dilution) of Mother Tincture (with proper respective) (e.g., a 1 part Mother tincture per 1,000,000,000,000,000,000,000,000,000,000 parts HPUS-approved diluent of 90% glycerin/10% purified water solution.)

The homeopathic element Silicon Dioxide (SiO₂) could be prepared into a HPUS-approved tincture by taking one part of silica, in powder, and four parts of dry sodium carbonate. The sodium carbonate could be fused in a large clay crucible. An appropriate amount of silica powder is then gradually added a portion at a time to the fused mass. During each addition, a noticeable amount of carbon dioxide escapes from the mixing. When the emission of carbon dioxide has ceased, pour the resulting combined mass upon a clean marble slab. After the mass had cooled but is still slightly warm transfer it to a mortar and break the mass into small pieces. Transfer the small pieces to a wide mouthed bottle, adding sufficient distilled water to dissolve mass. The bottle should be capped with a bladder.

On the next day, dilute the solution and rapidly filter it through cotton wool. Gradually add to the filtered solution, an appropriate amount of hydrochloric acid in small quantities. This should cause the silica to become hydrated and to precipitate out of the solution in the form of a bulky gelatinous white precipitate. Collect the precipitate and wash it with distilled water upon a square frame filter. The washing must be continued until the filtrate is tasteless and no longer precipitates a solution of silver nitrate. Then dry upon a porcelain water bath when filtrate shrinks to an impalpable powder.

The resulting filtration/titration could then be diluted using a HPUS-approved diluent of 90% glycerin/10% purified water solution utilizing a 1× dilution technique (with accompanying potentization). The resulting dilution could be a 13× dilution factor ( 1/10¹³ dilution) with a resulting dilution substantially being a 1 part tincture per 10,000,000,000,000 parts HPUS approved diluent of 90% glycerin/10% purified water solution. The Silicon Dioxide in the homeopathic composition could be thirteen times potency

The homeopathic ingredient Mercury could be prepared into a HPUS tincture by mixing in a suitable flask an appropriate amount of pure mercury with an appropriate amount of nitric acid and 235 ml of the purified water. The flask should then be gradually heated until about 70 g of the mercury has dissolved into solution. Taking a small portion of the solution and diluted with about twenty times its volume of purified water to yield a perfectly black precipitate with ammonia. Dilute the hot solution with 350 ml of purified water and while warm, filter it into a vessel containing four times its bulk of cold purified water. Having thoroughly mixed the filtrate with the purified water, add the solution of ammonia previously diluted with 290 ml of purified water in a thin stream, stirring constantly meanwhile, as soon as the precipitate has subsided, decant the supernatant liquid, shake the precipitate with a fresh portion of purified water, collect the precipitate on a filter, wash thoroughly and dry it between folds of filtering paper without the aid of heat.

The prepared mercury could then be triturated to solubility in mild lactose. This titration could then be diluted in a HPUS approved diluent of a 90% glycerin/10% purified water mix. This preparation could then be diluted thirteen (13) times utilizing a 1× dilution technique (with accompanying potentization). The resulting dilution could be a 13X dilution factor ( 1/10¹³ dilution) with a resulting dilution substantially being a 1 part preparation per 10,000,000,000,000 parts diluent (90% glycerin/10% purified water solution.) Mercury as an ingredient for the homeopathic composition of the invention could be at 13 times potency.

The homeopathic ingredient Salicylic Acid [C6H4(OH)COOH] could be prepared as a HPUS-approved Mother Tincture of 100 mg of salicylic acid dissolved in a sufficient amount of strong alcohol to produce 1000 ml of Mother Tincture. This tincture could then be diluted six (6) times utilizing a 1× technique dilution (with accompanying potentization). The resulting dilution could be a 6× dilution factor ( 1/10⁶ dilution) with a resulting dilution substantially being a 1 part preparation per 1,000,000 parts of diluent (90% glycerin/10% purified water solution.) The homeopathic ingredient salicylic acid could be at six times potency for the homeopathic composition of the invention.

For each 1× dilution of every active homeopathic ingredient, the resulting diluted volume could be potentized.

Once the proper tinctures are prepared, appropriate amounts of the prepared tinctures could be mixed into an appropriate amount of delivery matrix in proportions known by those with ordinary skill in the art. Delivery of the invention into the entrance of the ear canal may by of suitable means known to those skilled in the art, including but not limited to a medicine dropper or the like.

### CONCLUSION

In this manner, the homeopathic composition of the invention may be used to treat symptoms of hearing impairment due to aging of the subject. Such hearing impairment could include symptoms due to frequency-based sensorineural hearing loss; symptoms due to intensity-based sensorineural hearing loss, symptoms of tinnitus (including those occasioned by damage to the cilia of the hearing cells or by conventional medicine pharmaceuticals) and the like.

Although the description above contains many specifications, these should not be construed as limiting the scope of the invention but as merely providing illustrations of some of the presently preferred embodiments of this invention. Thus, the scope of the invention should be determined by the appended claims and their legal equivalents rather than by the examples given.

What is claimed is:

1. A homeopathic composition suitable for the treatment of the symptoms of hearing impairment, which comprises:
(A) 3-Isothiocyanato-1-Propene in 6 times potency,
(B) Arnica Montana in 6 times potency,
(C) Arnica Montana in 30 times potency,
(D) Calcium Sulphide in 6 times potency,
(E) Hypericum Perforatum in 6 times potency,
(F) Hypericum Perforatum in 30 times potency,
(G) Silicon Dioxide in 13 times potency,
(H) Mercury (Hg) in 13 times potency, and
(I) Salicylic Acid in 6 times potency.

2. A composition of claim 1 wherein the hearing impairment is tinnitus.

3. A composition of claim 2 wherein tinnitus is caused by damaged or otherwise impaired cilia of the hair cells.

4. A composition of claim 1 wherein composition may ameliorate symptoms of hearing impairment caused by damaged or otherwise impaired cilia of the hair cells.

5. A composition of claim 2 wherein the hearing impairment is frequency-based sensorineural hearing loss.

6. A composition of claim 1 wherein the hearing impairment is intensity-based sensorineural hearing loss.

7. A composition of claim 1 wherein the hearing impairment is caused by conventional medicine pharmaceuticals.

8. A method of treating the symptoms of hearing impairment by applying to the afflicted ear the following homeopathic composition comprising:
(A) 3-Isothiocyanato-1-Propene in 6 times potency,
(B) Arnica Montana in 6 times potency,
(C) Arnica Montana in 30 times potency;
(D) Calcium Sulphide in 6 times potency,
(E) Hypericum Perforatum in 6 times potency,
(F) Hypericum Perforatum in 30 times potency,
(G) Silicon Dioxide in 13 times potency,
(H) Mercury (Hg) in 13 times potency, and
(I) Salicylic Acid in 6 times potency.

9. A method of claim 8 wherein the hearing impairment is tinnitus.

10. A method of claim 8 wherein the tinnitus is caused by is caused by damaged or otherwise impaired cilia of the hair cells.

11. A method of claim 8 wherein the symptoms of hearing impairment are caused by damaged or otherwise impaired cilia of the hair cells.

12. A method of claim 8 wherein the symptoms of hearing impairment are caused by frequency-based sensorineural hearing loss.

13. A composition of claim 1 wherein the symptoms of hearing impairment are caused by intensity-based sensorineural hearing loss.

14. A composition of claim 8 wherein the symptoms of hearing impairment are caused by conventional medicine pharmaceuticals.
